(19) United States
(12) Patent Application Publication
(10) Pub. No.: US 2023/0084267 A1
(43) Pub. Date: Mar. 16, 2023

(54) SYSTEM AND A CONTROL METHOD THEREOF

(71) Applicant: CANON KABUSHIKI KAISHA, Tokyo (JP)

(72) Inventor: Koichi Tsujimoto, Kanagawa (JP)

(21) Appl. No.: 17/885,606

(22) Filed: Aug. 11, 2022

(30) Foreign Application Priority Data
Aug. 31, 2021 (JP) 2021-140655

(51) Int. Cl. G16H 50/20; G16H 70/60

(52) U.S. Cl. CPC G16H 50/20; G16H 70/60

(57) ABSTRACT

In a system that includes an evaluation server that uses a learning model to perform an evaluation of a disease name of an animal, a local terminal configured to request an evaluation of the name of a disease from the evaluation server, and a client terminal configured to display a result evaluated by the evaluation server, the local terminal is provided with a first transmission unit that transmits a request for a disease name evaluation that includes moving image data in which an animal is captured to the evaluation server. The evaluation server is provided with an evaluation unit that evaluates, in response to receiving a disease name evaluation request, a disease name from the moving image data by use of a learning model selected based on individual information of an individual that is a target of disease name evaluation, and a notification unit to notify the client terminal of the result of the evaluation by the evaluation unit. The client terminal is provided with a display unit that displays a notification page that includes the obtained result of the evaluation.

### BACKGROUND OF THE INVENTION

### Field of the Invention

The present invention relates to a system for detecting onset of disease in an animal and a control method thereof.

### Description of the Related Art

In recent years, systems using machine learning have been explored as a form of automating the health management of pets and livestock. In the health management of animals, tasks such as body weight measurement and exercise amount recording exist, but there are expectations for the automation of health diagnosis, particularly for the purpose of early detection of disease. This is because it is difficult for an specialist to be always present in a private home and there is a concern that a delay in detection could lead to increased severity of illness, and it is impractical for a livestock farmer to constantly monitor the health condition of all individual animals by visual observation. A common technique for the detection of disease onset in animals is a method in which sensors are attached to the body of an animal to measure body temperature and the like. However, the attachment of sensors to the body can be highly stressful for an animal, and it is difficult to attach sensors to small animals. For this reason, techniques for the detection of the onset of disease by using machine learning to analyze an image of a region in which the characteristics of a disease are highly visible are being studied. Japan Patent Application Laid-Open No. 2005-107768 discloses a technique of analyzing a facial image by machine learning and comparing the extracted features with disease patterns stored in a database to detect the onset of a disease.

However, in the technology of Japan Patent Application Laid-Open No. 2005-107768 for performing detection of a disease in an image of a specific body part such as a face, a symptom does not appear in a specific body part and a disease that cannot be detected in an image of the specific body part is not able to be detected. For example, in a case in which a disease is caused by the nervous system or a psychogenic disease caused by stress, because it is difficult to capture an image of a target, these diseases cannot be detected by a method that analyzes an image of a body part. In addition, many diseases caused by the nervous system and many psychogenic diseases are known to cause abnormal behavior at the onset of the disease. However, because the timing of this is dependent on a variety of elements, symptoms are not always apparent to the eyes of the animal's owner, and there are cases in which the detection of the disease may be delayed.

### SUMMARY OF THE INVENTION

The present invention provides a system for the detection of the onset of disease based on moving image data in which an animal is captured.

A system according to the present invention includes an evaluation server that uses a learning model to perform an evaluation of a disease name of an animal, a first information processing device that requests an evaluation of the name of a disease to the evaluation server, and a second information processing device that displays the result of the evaluation by the evaluation server, wherein the first information processing device is configured to include a first transmission device that transmits a request for a disease name evaluation that includes moving image data in which an animal is captured to the evaluation server, and wherein the evaluation server is configured to include an evaluation unit that evaluates, in response to receiving a disease name evaluation request from the first information processing device, a disease name from the moving image data by using learning model selected based on individual information of an individual that is a target of disease name evaluation, and a notification unit that notifies the second information processing device of a result of the evaluation by the evaluation unit, wherein the second information processing device is configured to include a display unit that displays a notification page that includes the result of the evaluation obtained from the evaluation server.

Further features of the present invention will become apparent from the following description of exemplary embodiments with reference to the attached drawings.

### BRIEF DESCRIPTION OF THE DRAWINGS

FIG. 1 is a diagram that shows an overall configuration of a disease name evaluation system.

FIG. 2 is a diagram that shows a hardware configuration of a disease name evaluation system.

FIG. 3 is a diagram that shows a software configuration of a disease name evaluation system.

FIG. 4 is a diagram that shows an example of an individual information registration page in the first embodiment.

FIG. 5 is a conceptual diagram that shows the relationship between a learning model and an input/output.

FIG. 6 is a flowchart that shows processing in which a learning model is generated.

FIG. 7 is a diagram that explains an operational sequence of a disease name evaluation system.

FIG. 8 is a flowchart that shows a disease name evaluation processing in the first embodiment.

FIG. 9 is a diagram that shows an example of a disease name notification page in the first embodiment.

FIG. 10 is a diagram that shows an example of an individual information registration page for registering individual information for individual identification in the second embodiment.

FIG. 11 is a flowchart that shows an individual information registration processing for individual identification according to the second embodiment.

FIG. 12 is a flowchart that shows a disease name evaluation processing according to the second embodiment.

### DESCRIPTION OF THE EMBODIMENTS

### First Embodiment

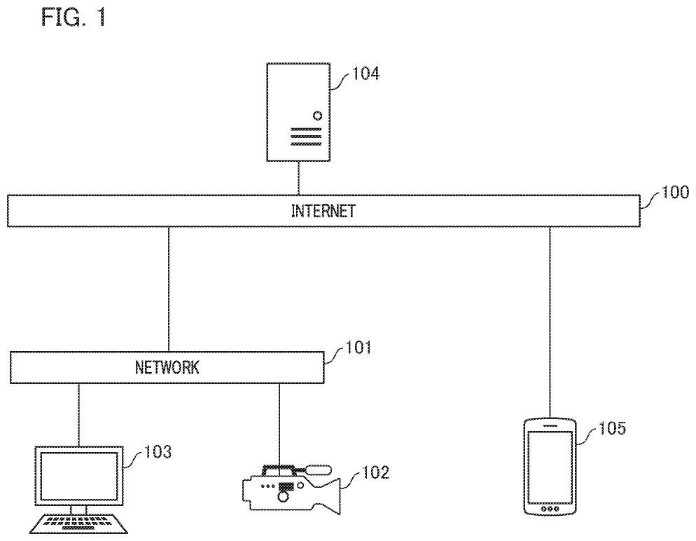

FIG. 1 is a diagram showing an overall configuration of a disease name evaluation system according to the present embodiment. The disease name evaluation system includes an evaluation server 104, a video camera 102, a local terminal 103, and a client terminal 105. In the disease name evaluation system, the moving image data captured by the video camera 102 is sent to the local terminal 103, and the local terminal 103 transmits a disease name evaluation request that includes the moving image data to the evaluation server 104. The evaluation server 104 that has performed a disease name evaluation in response to a disease name evaluation request transmits the result of the evaluation to the local terminal 103 and the client terminal 105.

The evaluation server 104, the video camera 102, the local terminal 103, and the client terminal 105 are communicatively coupled to each other via a network. Specifically, the local terminal 103 and the video camera 102 are connected via a network 101, which is a local network. The network 101, the evaluation server 104, and the client terminal 105 are connected via the network 105 are connected via a network 100. The network 100 is, for example, the Internet. Note that, in the present embodiment, although an example in which the network 101 is a local network and the network 100 is the Internet is explained, the configuration of a network is not limited thereto. The network 100 and the network 101 may be configured by a communication network such as a local area network (LAN) or a wide area network (WAN), a cellular network (for example, a Long Term Evolution (LTE) network, a 5th Generation (5G) network, or the like), a wireless network, a telephone line, a dedicated digital line, or a combination thereof. Thus, the network 100 and the network 101 need only be configured to transmit and receive data, and any method of communication can be adopted.

The video camera 102 is an image capturing device that captures moving image data of an individual animal that is the target of the disease name evaluation. In addition, the video camera 102 includes a network connection function, and transmits the captured moving image data to the local terminal 103. Further note that, in the present embodiment, although an example of capturing an image of an animal with the video camera 102 is explained, the device for capturing an image of an animal may be any device other than a video camera, provided that such a device includes hardware and software for capturing moving image data. For example, a client terminal that includes a camera function, such as a smartphone, may be used. Note that, in the present embodiment, although an example in which the video camera 102 includes a communication function via a network is explained, the moving image data captured by the video camera 102 may be obtained by the local terminal 103, and the video camera 102 does not need to include a communication function. For example, the video camera 102 and the local terminal 103 may be directly connected by a cable or the like to transmit moving image data, and the video camera 102 may be configured to read a medium on which moving image data has been stored by the local terminal 103.

The local terminal 103 is an information processing device that includes a built-in program execution environment, and is, for example, a desktop personal computer, a notebook personal computer, a tablet terminal, a Personal Data Assistant, a smartphone, and the like. The local terminal 103 obtains the captured moving image data of the individual animal that is the target of the disease name evaluation from the video camera 102, and transmits a request for the disease name evaluation based on the moving image data to the evaluation server 104. In addition, the local terminal 103 receives the registration of individual information to be used for disease name evaluation and transmits the registered individual information to the evaluation server 104. Note that if the local terminal 103 includes hardware and software that captures moving image data, the video camera 102 and the local terminal 103 may be configured as a single unit. At this time, the captured moving image data is directly transmitted to the evaluation server 104.

The evaluation server 104 provides a service that evaluates the disease name of the animal based on the moving image data and the individual information of the individual animal. In the present embodiment, the evaluation server 104 evaluates the disease name of the animal based on the individual information and the moving image data received from the local terminal 103. In addition, the evaluation server 104 transmits the evaluated result to the local terminal 103 and the client terminal 105. Note that in addition to one or a plurality of information processing devices, the evaluation server 104 may be realized by a virtual machine (cloud service) that uses a resource provided by a data center that includes an information processing device, or by a combination thereof

The client terminal 105 is an information processing device, such as a smartphone, that includes a browser function for browsing data on a Web server via a Web browser (software provided for use of the World Wide Web) or the like. The client terminal 105 of the present embodiment is connected to the network 100, ad enables the browsing of the data provided by the evaluation server 104.

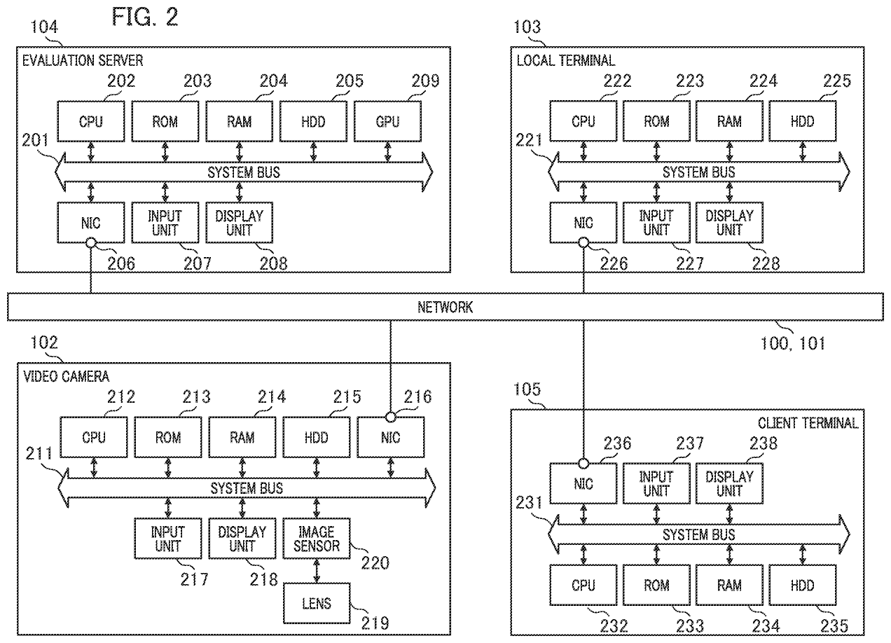

FIG. 2 is a diagram showing an example of a hardware configuration of each constituent element of the disease name evaluation system. First, the hardware configuration of the evaluation server 104 will be explained. The evaluation server 104 includes a CPU 202, a ROM 203, a RAM 204, an HDD 205, an NIC 206, an input unit 207, a display unit 208, and a GPU 209, which are connected to each other by a system bus 201. Note that in the present embodiment, although an example of the case in which one CPU executes each of the processes by using one memory is shown to simplify the explanation of the evaluation server 104. However, the evaluation server 104 may be otherwise configured. For example, a plurality of processors, RAMs, ROMs, and storages can cooperate and execute the each of the processes shown in the flowcharts explained below. Further, resources of a plurality of server computers can cooperate with each other to achieve each of a service.

A Central Processing Unit (CPU) 202 comprehensively controls access of each constituent element connected to the system bus 201, and controls the entire device. The Read Only Memory (ROM) 203 is a storage unit, and stores various data such as a basic I/O program therein. The Random Access Memory (RAM) 204 is a temporary storage unit, and functions as the main memory, a work area, or the like of the CPU 202 and the GPU 209. A Hard Disk Drive (HDD) 205 is one of the storage units that functions as a large-capacity memory and stores, for example, an application program and the like. Note that in the present embodiment, the HDD 205 is explained as an example of a storage unit, but the present invention is not limited thereto, and the storage unit may be a Solid State Drive (SSD), or a device that can read/write data by loading an external medium such as a memory card.

A Network Interface Card (NIC) 206 performs exchange of data with the local terminal 103, the client terminal 105, or the like that are connected via the network 100. The input unit 207 receives an instruction/input from a user via an input device such as a keyboard (not shown) or a mouse (not shown). The display unit 208 includes an output device such as a display (not shown) and displays various data to the user.

The Graphics Processing Unit (GPU) 209 performs processing to perform learning over a plurality of times by the use of a learning model, such as deep learning. Using the GPU 209 enables the performing of parallel processing of a greater amount of data, thus attaining efficient computation. Note that the configuration of the evaluation server 104 eplained in the present embodiment is simply an example, and is not limited thereto. For example, the storage destination of data and programs can be changed to any of the ROM 203, the RAM 204, and HDD 205 in accordance with the features of the data and programs.

Next, the hardware configuration of the video camera 102 will be explained. The video camera 102 includes a CPU 212, a ROM 213, a RAM 214, an HDD 215, a NIC 216, an input unit 217, a display unit 218, an image sensor 220, and a lens 219. The CPU 212 to the image sensor 220 are connected to each other by a system bus 211.

The CPU 212 comprehensively controls access of each constituent element connected to the system bus 211 and controls the entire device. The ROM 213 is a storage unit, and stores various data such as a basic I/O program therein. The RAM 214 i a temporary storage unit, and functions as the main memory, a work area, or the like of the CPU 212.

The HDD 215 is one of the storage units and functions as a large-capacity memory, and stores an application program and the moving image data read by the image sensor 220. Note that in the present embodiment, the HDD 215 is explained as an example of a storage unit, but the present invention is not limited thereto, and for example, the HDD 215 may be a device that can read/write data by loading an external medium such as a memory card. The NIC 216 performs data exchange with the local terminal 103 via the network 101. The input unit 217 receives an instruction/input from the user via an input device, such as a hardware button (not shown). The display unit 218 includes an output device such as a display (not shown), and displays various data to the user.

The lens 219 is an image capturing optical system that includes plurality of lenses such as a shift lens or a zoom lens, and an aperture. The lens 219 forms an optical image on the image sensor 220. The image sensor 220 is an image capturing unit that includes a photoelectric conversion element, such as a CMOS or a CCD, and that outputs an output signal corresponding to an optical image. The image data and the moving image data acquired by image capture by the image sensor 220 are stored on the HDD 215. The image data and the moving image data stored on the HDD 215 are transmitted to the local terminal 103 via the NIC 216. Note that the moving image data may be directly transmitted from the video camera 102 to the evaluation server 104 without going through the local terminal 103.

Note that the configuration of the video camera 102 is not limited to the configuration explained above. For example, the storage destination of data and programs can be changed to any of the ROM 213, the RAM 214, or HDD 215 in accordance with the features of the data and programs. In addition, the image data and the moving image data are stored in an external storage device such as a memory card, and may be transmitted to the local terminal 103 by reading the external storage device with the local terminal 103.

Next, the hardware configuration of the local terminal 103 and the client terminal 105 will be explained. Because the local terminal 103 and the client terminal 105 have a similar hardware configuration, only the local terminal 103 will be explained herein. The local terminal 103 includes a CPU 222, a ROM 223, a RAM 224, an HDD 225, an NIC 226, an input unit 227, and a display unit 228, which are connected to each other by a system bus 221.

The CPU 222 comprehensively controls access of each constituent element connected to the system bus 221, and performs control of the entire device. The ROM 223 is a storage unit, and stores various data such as a basic I/O program therein. The RAM 224 is a temporary storage unit, and functions as the main memory, a work area, or the like of the CPU 222.

The HDD 225 functions as a large-capacity memory in one of the storage units, and stores a program and various data. For example, moving image data acquired from the video camera 102 is also stored on the HDD 225. Note that in the present embodiment, the HDD 225 is explained as an example of a storage unit, but the present invention is not limited thereto, and for example, the storage unit may be a device that can read/write data by loading an external medium such as a memory card.

The NIC 226 performs the exchange of data with an external device such as the video camera 102 and the evaluation server 104 via the network 100 and the network 101. The input unit 227 receives an instruction/input from a user via an input device such as a keyboard (not shown), or a mouse (not shown). The display unit 228 includes an output device such as a display (not shown) and displays various data to the user. The input unit 227 and the display unit 228 may be integrally configured as a touch panel or the like. By associating the input coordinates and the display coordinates on the touch panel, it is possible to configure a GUI such that the user can directly operate the screen displayed on the touch panel.

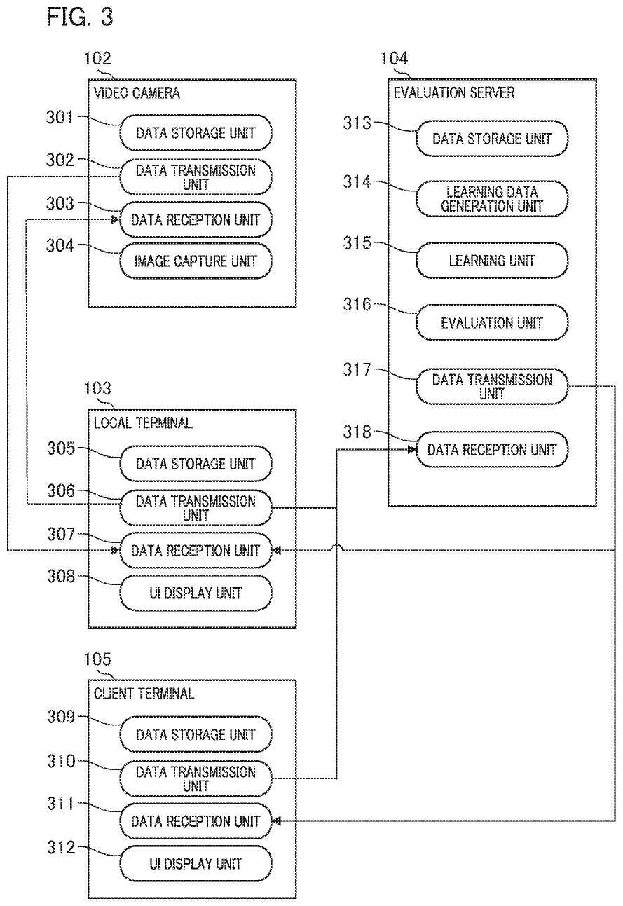

FIG. 3 is a diagram showing an example of a software configuration of the disease name evaluation system. First, a software configuration of the video camera 102 will be explained. The video camera 102 includes a data storage unit 301, a data transmission unit 302, a data reception unit 303, and an image capture unit 304. The image capture unit 304 converts light input via the lens 219 into a signal by the image sensor 220, and acquires the moving image data. Data storage unit 301 stores and manages the moving image data acquired by the image capture unit 304 on the HDD 215. The data reception unit 303 receives instructions (requests) from the local terminal 103 connected via the NIC 216. For example, the data reception unit 303 receives an image capture start request and an image capture stop request from the local terminal 103. The moving image data captured in accordance with the request received from the local terminal 103 is stored on the HDD 215 by the data storage unit 301. The data transmission unit 302 transmits data to the local terminal 103 connected via the NIC 216. For example, the data transmission unit 302 transmits the moving image data stored on the HDD 215 to the local terminal 103.

Next, a software configuration of the local terminal 103 will be explained. The local terminal 103 includes a data storage unit 305, a data transmission unit 306, a data reception unit 307, and a UI display unit 308. The data storage unit 305 stores various data such as moving image data, individual information, and disease name evaluation results. In the present embodiment, the data storage unit 305 stores the moving image data received from the video camera 102 on the HDD 225 in association with the individual information of the moving image to be captured.

The data transmission unit 306 transmits data and an instruction (request) to an external device, such as the evaluation server 104 or the video camera 102, via the NIC 206. For example, the data transmission unit 306 transmits an image capture start request and an image capture stop request to the video camera 102. In addition, the data transmission unit 306 (first transmission unit) transmits the individual information and the moving image data stored on the HDD 225 together with the disease name evaluation request to the evaluation server 104. The data reception unit 307 receives data from an external device such as the evaluation server 104 or the video camera 102 via the NIC 206. For example, the data reception unit 307 receives moving image data from the video camera 102. The received moving image data is stored on the HDD 225 by the data storage unit 305. In addition, the data reception unit 307 receives a disease name evaluation result from the evaluation server 104. The received disease name evaluation result is stored on the HDD 225 by the data storage unit 305.

The UI display unit 308 controls display to the display unit 228, and receives an input by the user to the displayed UI. For example, the UI display unit 308 displays on the display unit 228 an individual information registration page for input of information on an individual that is the target of the disease name evaluation. Here, the individual information registration page will be explained with reference to FIG. 18.

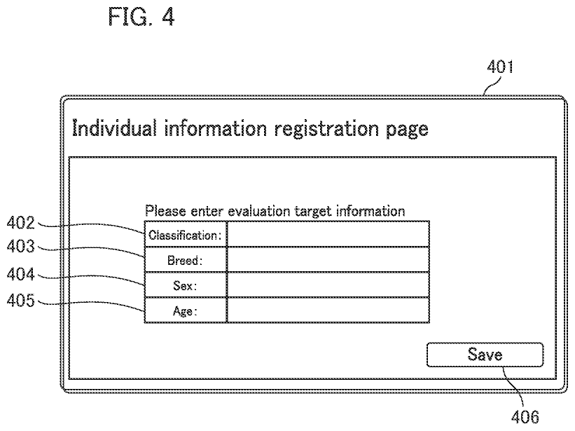

FIG. 4 is a diagram showing an example of an individual information registration page. The individual information registration page is a screen for registering in advance the individual information to be included in the disease name evaluation request in the local terminal 103. An individual information registration page 401 is displayed on the display unit 228 by the UI display unit 308 of the local terminal 103. The individual information registration page 401 displays, as forms for inputting individual information, a classification input form 402, a breed input form 403, a sex input form 404, and an age input form 405. Note that these are examples of forms displayed on the individual information registration page 401, and are not limited thereto.

The classification input form 402 is a form into which the type of the classification of animal that is the target individual of the disease name evaluation is input. The breed input form 403 is a form to further input what breed the individual that is the target of the disease name evaluation is from among the animal species that was input in the classification input form 402. The sex input form 404 is a form to input the sex (female or male) of the individual that is the target of the disease name evaluation. The age input form 405 is a form to input the age of the individual that is the target of the disease name evaluation. In addition, a save button 406 is displayed in the individual information registration page 401. When the save button 406 is pressed by the user, the information input in each form (classification input form 402 to age input form 405) is stored as individual information on the data storage unit 305. Table 1 is an example of individual information.

TABLE 1

| Individual ID | Classification | Breed | Sex | Age |
|---|---|---|---|---|
| neko001 | Cat | Crossbreed | Male | 3 |

Individual information includes, for example, an individual ID, a classification, a breed, a sex (female or male), and an age. The individual ID is information for the uniquely identifying an individual. Classification is information that indicates the broad species of an individual, such as dog, cat, horse, cow, or chicken. For example, in the case of a cat, breed is information indicating the breed, such as British Shorthair, Munchkin, Persian, Tortoiseshell, or crossbreed. In the present embodiment, as the individual information, the “classification,” “breed,” “sex,” and “age” of the individual that is the target of an image capture that is input by the user on the individual information registration page, is managed in association with the individual ID. However, the items of the individual information shown in Table 1 are simply examples, and are not limited thereto.

In addition, the UI display unit 308 displays the disease name evaluation result received from the evaluation server 104 and stored in the data storage unit 305 on the display unit 228. Table 2 is an example of a disease name evaluation result.

TABLE 2

| Evaluation | | | | | Evaluation |
|---|---|---|---|---|---|
| Request ID | Classification | Breed | Sex | Age | Result |
| neko001 | Cat | Crossbreed | Male | 3 | Disease A |

A disease name evaluation result includes individual information and a disease name evaluation result, in addition to an “evaluation request ID” that uniquely identifies the disease name evaluation request. The evaluation request ID may be the same ID as that of the individual ID. An evaluation result is a result of the evaluation of a disease by the evaluation server 104 based on moving image data and individual information according to a disease name evaluation request.

Next, a software configuration of the client terminal 105 will be explained. The client terminal 105 includes a data storage unit 309, a data transmission unit 310, a data reception unit 311, and a UI display unit 312. The data reception unit 311 receives data from an external device such as the evaluation server 104 via the NIC 206. For example, data reception unit 311 receives a disease name evaluation result from the evaluation server 104. The received disease name evaluation result is stored on the HDD 235 by the data storage unit 309.

The UI display unit 312 controls display to a display unit 238, and in addition, receives an input by the user to the displayed UI. For example, the UI display unit 312 displays the disease name evaluation results (Table 2) received from the evaluation server 104. In addition, the UI display unit 312 displays the information extracted from the disease name evaluation result on a disease name notification page 900 to be described below. Further, the UI display unit 312 receives an input of a feedback result from the user on the disease name notification page 900. The feedback result is a result of an analysis by a veterinarian with respect to the individual that is the target of the disease name evaluation.

The data storage unit 309 stores various data such as disease name evaluation results. In the present embodiment, the data storage unit 309 stores the disease name evaluation results received from the evaluation server 104 on the HDD 235. In addition, the data storage unit 309 stores the feedback result input by the user on the disease name notification page 900 in association with the disease name evaluation result on the HDD 224 as feedback information. Table 3 is an example of feedback information.

TABLE 3

| Evaluation | Classi- | | | | Evaluation | Diagnosis |
|---|---|---|---|---|---|---|
| Request ID | fication | Breed | Sex | Age | Result | Result |
| neko001 | Cat | Cross- | Male | 3 | Disease A | Healthy |
| | | breed | | | | |

Feedback information includes a disease name evaluation result (Table 2) and a diagnosis result provided by an specialist that the user input with respect thereto. In the example of Table 3, because the user input “healthy” as a diagnosis result with respect to the “Disease A” evaluation result, it can be understood that the disease name evaluation result is incorrect. In a case in which the disease name evaluation result is correct, the value of the “Evaluation Result” in Table 3 and the value of the “Diagnosis Result” (disease name) are the same. The data transmission unit 310 transmits data to an external device such as the evaluation server 104 via the NIC 206. For example, the data transmission unit 310 transmits the feedback information (Table 3) stored on the HDD 224 to the evaluation server 104.

Next, a software configuration of the evaluation server 104 will be explained. The evaluation server 104 includes a data storage unit 313, a learning data generation unit 314, a learning unit 315, an evaluation unit 316, a data transmission unit 317, a data transmission unit 317, and a data reception unit 318. The data storage unit 313 stores various data such as moving image data, individual information, feedback information, a learning model, and behavior data. In the present embodiment, the data storage unit 313 stores the moving image data and the individual information received from the local terminal 103, and a learning model on the HDD 224. Further, the data storage unit 313 stores the feedback information received from the client terminal 105 on the HDD 224.

The learning data generation unit 314 generates learning data based on moving image data and feedback information (Table 3). Specifically, the learning data generation unit 314 first obtains the classification of the diagnosis target (Table 4 to be explained later) from the individual information (Table 1) stored on the HDD 224. Then, the learning data generation unit 314 extracts time-series data (hereinafter referred to as “behavior data”) of the position information of each body part of the diagnosis target from the moving image data, and further associates the disease name with the individual information, and generates the learning data. Note that the procedure and method for the extraction of behavior data from the moving image data have no direct relation to the content of the present invention, and therefore an explanation thereof shall be omitted. Further, the learning data generation unit 314 extracts the classification, the breed, the age, the gender, and the diagnosis results from the feedback information stored on the HDD 224, and generates new learning data. The generated learning data is associated with the behavior data and the evaluation request ID obtained from the HDD 224, and used in the next learning processing.

The learning unit 315 performs learning by using the learning data generated by the learning data generation unit 314, and generates and educates (updates) a learning model. Specifically, the behavior data (moving image data) and disease names are used to generate a learning model for disease name evaluation based on the behavior data. The learning model may be generated for each classification of individual information, or may be generated for each breed, gender, or age. The evaluation unit 316 evaluates a disease name based on the moving image data received from the local terminal 103. The evaluation unit 316 performs disease name evaluation by the execution of a disease name evaluation program by using a learning model. The learning model used in the evaluation by the evaluation unit 316 is selected based on the individual information.

The data transmission unit 317 transmits data to an external device such as the local terminal 103, the client terminal 105, or the like, via the NIC 206. For example, the data transmission unit 317 transmits the disease name evaluation result to the local terminal 103 and the client terminal 105. The data reception unit 318 receives data from an external device such as the local terminal 103 and client terminal 105 via the NIC 206. For example, the data reception unit 318 receives a disease name evaluation request that includes the individual information and the moving image data from the local terminal 103. In addition, the data reception unit 318 receives the feedback information from the client terminal 105.

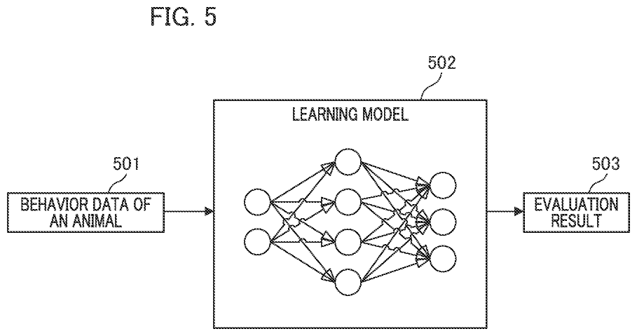

FIG. 5 is a conceptual diagram showing the relationship between the learning model 502 used in the evaluation server 104 and the input/output. The learning model 502 is generated by the learning unit 315 of the evaluation server 104, and used in the disease name evaluation program executed by the evaluation unit 316. The learning model 502 machine learns by using animal behavior data. Specific examples of algorithms for machine learning include the nearest-neighbor method, the naive Bayes method, and support vector machine. Further, deep structured learning (deep learning) which utilizes a neural network to generate locally feature values and combining weighting factors for learning may also be given. Algorithms that can be utilized among the above described algorithms can be appropriately used and applied to the present embodiment.

The behavior data 501 of an animal, which is the data input to the learning model 502, is the behavior data generated by analyzing the moving image data that captured an individual animal received from the local terminal 103. A disease name evaluation result 503, which is the data output by the learning model 502, is a result of the evaluation of the disease name of the individual corresponding to the input behavior data. In a case in which the disease name can be evaluated, the learning model 502 outputs the disease name as the disease name evaluation result 503. Note that the learning model 502 may output a probability that is a disease of the disease name evaluation result 503 together with the disease name evaluation result 503.

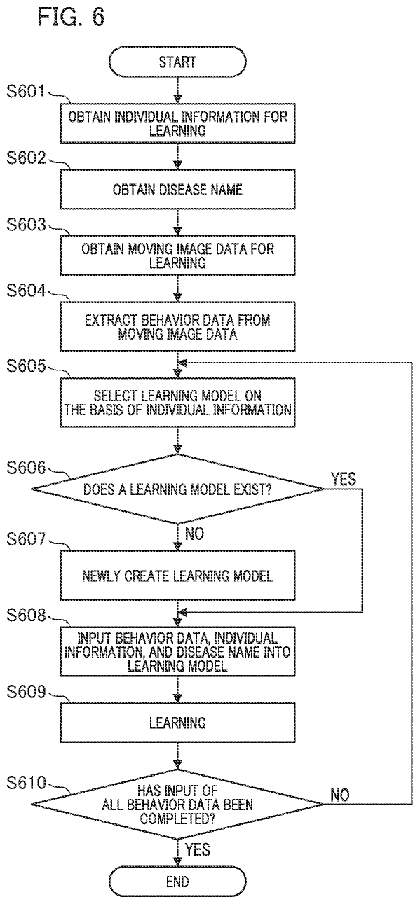

The learning phase of the learning model 502 will be explained. FIG. 6 is a flowchart that shows processing in which the learning unit 315 of the evaluation server 104 generates the learning model 502. Each of the processes shown in FIG. 6 is implemented by the CPU 202 and the GPU 209 of the evaluation server 104 loading a program that is stored on the ROM 403 or the HDD 205 onto the RAM 204 and executing the program.

In step S601, the learning unit 315 obtains the individual information for learning (hereinafter referred to as “individual information for learning”) that is stored in the data storage unit 313 in advance. Table 4 is an example of individual information for learning.

TABLE 4

| Individual | | | | | |
|---|---|---|---|---|---|
| ID for | | | | | Evaluation |
| learning | Classification | Breed | Sex | Age | Result |
| study001 | Cat | Crossbreed | Male | 3 | Disease A |

The individual information for learning includes individual information and a disease name of the individual thereof. Specifically, the individual information for learning includes as individual information, an individual ID for learning that uniquely identifies the individual information for learning, classification, breed, sex, age, and disease name.

In step S602, the learning unit 315 obtains a disease name from the individual information for learning obtained in step S601. In step S603, the learning unit 315 obtains moving image data for learning associated with the individual information for learning. The moving image data for learning is moving image data in which behavior specific to the disease of an individual animal in which a disease name has been identified by an specialist has been captured. For example, moving image data for learning is associated with individual information for learning by an individual ID for learning for identification. Table 5 is an example of moving image data for learning.

TABLE 5

| Individual ID for learning | Moving image file |
|---|---|
| study001 | Cat_diseaseA001.avi |

The moving image data for learning includes the individual ID for learning which is used for association with the individual information for learning and the moving image file.

In step S604, the learning data generation unit 314 extracts the behavior data for learning from the moving image data for learning obtained in step S603. In addition, the learning data generation unit 314 generates learning data in which the extracted behavior data for learning is associated with the disease name obtained in step S602. In step S605, the learning unit 315 selects a learning model based on the individual information for learning (Table 4). The selection of the learning model based on the individual information for learning will be explained with reference to Table 6.

Table 6 is an example of a unit that generates a learning model. A learning model may be generated for each disease, or may be generated for each combination of a disease and information included in individual information. For example, in the example of Table 6, a learning model is generated for each classification, age, and disease. In the case of the selection of a learning model according to the individual information for learning shown in Table 4, because the classification is “cat”, the age is “3”, and the name of the disease is “Disease A”, the learning model “Cat_3-5_years_old_Learning_model_for_Disease_A” among the learning models shown in Table 6 is selected. Note that a learning model may be generated for each classification of the individual information (Table 1), or further may be generated for each breed, sex, and age.

TABLE 6
Learning model name

Cat_3-5_years_old_Learning_model_for_Disease_A
Cat_6-10_years_old_Learning_model_for_Disease_A
Dog_0-2_years_old_Learning_model_for_Disease_B

In step S606, the learning unit 315 determines whether the learning model selected in step S605 exists. In the case that the learning model selected in step S605 exists, the processing proceeds to step S608. In contrast, in the case that the learning model selected in step S605 does not exist, the processing proceeds to step S607. In step S607, the learning unit 315 newly creates a learning model.

In step S608, the learning unit 315 inputs the behavior data for learning and the disease name extracted in step S604 to the learning model selected in step S605 or the learning model newly created in step S607. The behavior data for learning is used as input data, and the disease name is used as training data in each of the learning models.

In step S609, the learning unit 315 performs machine learning processing of the learning model. Specific examples of algorithms for machine learning include the nearest-neighbor method, the naive Bayes method, decision tree, and support vector machine. Further, deep structured learning (deep learning) which utilizes a neural network to generate locally feature values and combined weighting coefficients for learning, is another example. Algorithms that can be utilized among the above described algorithms can be appropriately used and applied to the present embodiment. Because the GPU 209 can perform more efficient operations by processing more data in parallel, when a learning model such as deep learning is used to perform learning over a plurality of times, it is it is effective to perform processing on the GPU 209. Thus, in processing by the learning unit 315, the GPU 209 is used in addition to the CPU 202 for processing. Specifically, in the case of executing a learning program that includes a learning model, learning is performed by the CPU 202 and the GPU 209 cooperatively performing computational operations. Note that operation of the learning unit 315 may be performed by only the CPU 202 or the GPU 209. Further, the evaluation unit 316 may also use the GPU 209 similarly to the learning unit 315.

In step S610, the learning unit 315 determines whether all of the behavior data for learning that was extracted in step S604 has been used. In a case in which all behavior data for learning has been used, the learning processing is terminated. In a case in which behavior data for learning remains, the processing returns to step S605, and the processing of steps S605 through S610 is repeated.

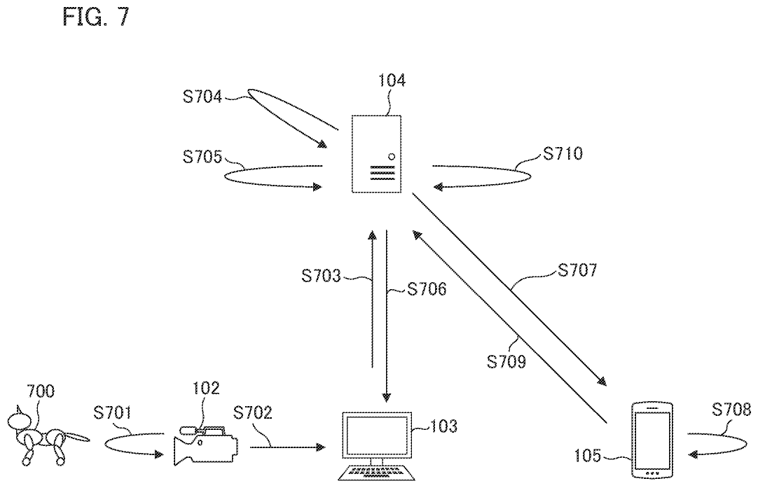

FIG. 7 is a diagram that explains an operational sequence of the disease name evaluation system. In the disease name evaluation system, the learning model 502 shown in FIG. 5 is used. In step S701, the video camera 102 captures moving image data of an individual animal 700 that is the target of the disease name evaluation. In step S702, the video camera 102 transmits the captured moving image data to the local terminal 103. The local terminal 103 that received the moving image data stores the moving image data in the data storage unit 305.

In step S703, the local terminal 103 transmits the disease name evaluation request that includes the stored moving image data and the individual information of the animal 700 to the evaluation server 104. In step S704, the evaluation server 104 analyzes the received moving image data and creates behavior data. In step S705, the evaluation server 104 selects a learning model based on the received individual information, and performs disease name evaluation processing by inputting the behavior data created in step S704 to the selected learning model, and. Upon completion of the evaluation processing in all the learning models selected in step S705, the processing proceeds to step S706. In step S706, the evaluation server 104 transmits the disease name evaluation result to the local terminal 103. The local terminal 103 that has received the disease name evaluation result stores the disease name evaluation result in the data storage unit 305, and the disease name evaluation result is displayed on the display unit 228 in accordance with a user operation.

In a case in which one or more diseases has been detected in the disease name evaluation processing of step S705, in step S707, the evaluation server 104 transmits the moving image data and the disease name evaluation results to the client terminal 105. In this case, the evaluation server 104 may transmit moving image data directly to the client terminal 105, or it may upload the data to an external server and transmit a URL to the client terminal 105 for the purpose of viewing the uploaded moving image data.

In step S708, the client terminal 105 displays on the display unit 238 a disease name notification page 900 that includes the results of the disease name evaluation received from the evaluation server 104. The disease name notification page 900 is described below. In a case in which the user presses a feedback transmission button 906 in the disease name notification page 900, the processing transitions to step 609. In contrast, in a case in which the user does not press the feedback transmission button 906 in the disease name notification page 900, the series of processing of the disease name evaluation is terminated.

In step S709, the client terminal 105 transmits the feedback information input by the user to evaluation server 104. In step S710, the evaluation server 104 stores the feedback information received from the client terminal 105. The stored feedback information is used as individual information for learning in a separate re-learning processing.

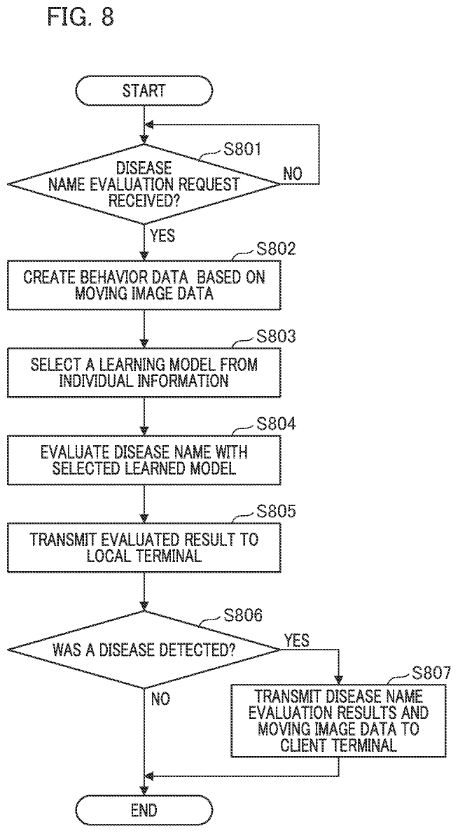

FIG. 8 is a flowchart that shows the disease name evaluation processing in step S705. Each of the processes shown in FIG. 8 is realized by the CPU 202 and the GPU 209 of the evaluation server 104 loading a program that is stored on the ROM 403 or the HDD 205 into the RAM 204, and executing the program.

In step S801, the data reception unit 318 determines whether a disease name evaluation request that includes individual information and moving image data of the individual that is the target of the disease name evaluation has been received from the local terminal 103. In a case in which a disease name evaluation request is received, the processing proceeds to step S802. In contrast, in a case in which a disease name evaluation request has not been received, the processing waits until one is received.

In step S802, the evaluation unit 316 creates behavior data based on the moving image data in the disease name evaluation request. In step S803, the evaluation unit 316 selects a learning model 502 to be used based on the individual information included in the disease name evaluation request. The learning model 502 may be selected based on the classification of individual information, or it may be selected based on breed, sex, age, or a combination thereof

In step S804, the evaluation unit 316 performs the disease name evaluation processing by using the learning model 502 selected in step S803. The evaluation unit 316 inputs the behavior data created in step S802 to the learning model 502, performs the processing of the disease name evaluation, and outputs the disease name evaluation result. In step S805, the data transmission unit 317 transmits the disease name evaluation result to the local terminal 103. In step S806, the data transmission unit 317 determines whether or not a disease has been detected in the disease name evaluation. In a case in which a disease is not detected, the disease name evaluation processing has been terminated. In contrast, in a case in which a disease is detected, the processing proceeds to step S807.

In step S807, the data transmission unit 317 transmits the moving image data obtained from the disease name evaluation request and the disease name evaluation result (disease name) to the client terminal 105. For example, the data transmission unit 317 transmits the URL of a disease name notification page 900 that includes the disease name evaluation result and the moving image data by e-mail. In addition, the URL of the disease name notification page 900 may be transmitted by push notification from the evaluation server 104. Note that moving image data and a disease name evaluation result may be periodically retrieved on the client terminal 105 side by Javascript running on the disease name notification page 900. In addition, information such as a percentage indicating the likelihood of the disease together with the disease name evaluation result may be transmitted to the client terminal 105.

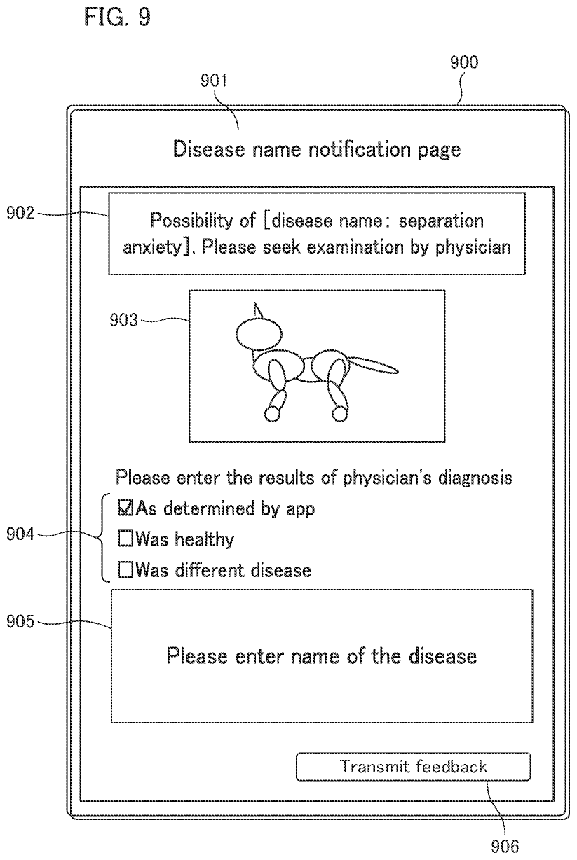

FIG. 9 is a diagram that shows an example of a disease name notification page. The disease name notification page 900 is displayed on the display unit 238 by the UI display unit 312 of the client terminal 105 in step S708. The disease name notification page 900 displays, for example, a page title 901, a disease name notification message 902, a moving image display region 903, a diagnosis result input checkbox 904, a disease name input form 905, and a feedback transmission button 906.

A page title 901 is a title that indicates that a page being displayed is a disease name notification page. The disease name notification message 902 is a character string for disease name notification generated by combining a character string for a notification message stored in the data storage unit 309 and a disease name received from the evaluation server 104. The moving image display region 903 is a region configured to display moving image data of the individual that is the target of disease name evaluation received from the evaluation server 104. The moving image display region 903 is provided in a case in which a symptom of an individual is not exhibited when the individual is examined by an specialist.

The diagnosis result input checkbox 904 is a checkbox for inputting a diagnosis result by an specialist. The diagnosis result input checkbox 904 displayed is, for example, a checkbox that is displayed in a case in which the disease evaluation result is consistent with a diagnosis by an specialist, in a case in which the disease evaluation result differs from the specialist and the patient is healthy, and in a case in which the disease evaluation result differs from the specialist and a different disease has been diagnosed. The user compares the disease evaluation result by the disease evaluation system with the diagnosis result by an specialist, and selects the appropriate check box to be checked. In a case in which a “Was different disease” checkbox is selected (that is, in a case in which a disease that is different from the disease name evaluation result by the evaluation server 104) is diagnosed, the disease name input form 905 is enabled. The disease name input form 905 is a region in which the name of the disease diagnosed by an specialist is input. In contrast, in a case in which the checkbox “As determined by app” is selected (that is, in a case in which this is the same as the disease name evaluation result by the evaluation server 104), or the case in which the checkbox “Was healthy” is selected, the disease name entry form 905 is in a disabled state. When the feedback transmission button 906 is pressed by the user, the data transmission unit 310 transmits the feedback information (Table 3) in accordance with the content of the diagnosis result input checkbox 904 and the disease name input form 905 to the evaluation server 104. The feedback information is used to train the learning model in the evaluation server 104. Note that, although the present embodiment explains an example of performing feedback by displaying the disease name notification page 900 by the client terminal 105, this may be configured to perform feedback by displaying the disease name notification page 900 by the local terminal 103.

As described above, according to the present embodiment, by collecting data analyzed and extracted from moving image data and using the learning results as a learning model on the evaluation server 104, it is possible to automatically detect the onset of disease without the need to go to the location of the animal or be touched by the animal. In addition, by collecting the diagnosis result from specialists as feedback, the accuracy of the evaluation result can be improved.

### Second Embodiment

In the system explained in the first embodiment, behavior data is extracted from the moving image data based on the information of one (1) individual animal for which the user has input individual information on the individual information registration page 401. However, in a case in which a plurality of animals are captured in the moving image data, such as in a case in which many pets are kept or in livestock breeding, the behavior data may be mixed, and the accuracy of the disease name diagnosis may be reduced. Therefore, in a second embodiment, a disease name evaluation system that detects the onset of disease while maintaining accuracy even in a case in which a plurality of animals are displayed in the moving image data will be explained. Note that, because the basic configuration and processing are similar to those of the first embodiment, only differences from the first embodiment are explained.

In the second embodiment, the individual information for individual identification is registered in advance in the evaluation server 104 to enable the identification of an individual animal among a plurality of animals from moving image data. Individual information for individual identification is information in which individual information and a feature amount for the identification of an individual in a moving image (“feature amount for individual identification”) are associated with each other. In order to register individual information for individual identification in the evaluation server 104, the user inputs individual information on the individual information registration page at the local terminal 103. In addition, the data transmission unit 306 (second transmission unit) of the local terminal 103 transmits an individual information registration request that includes individual information for individual identification to the evaluation server 104.

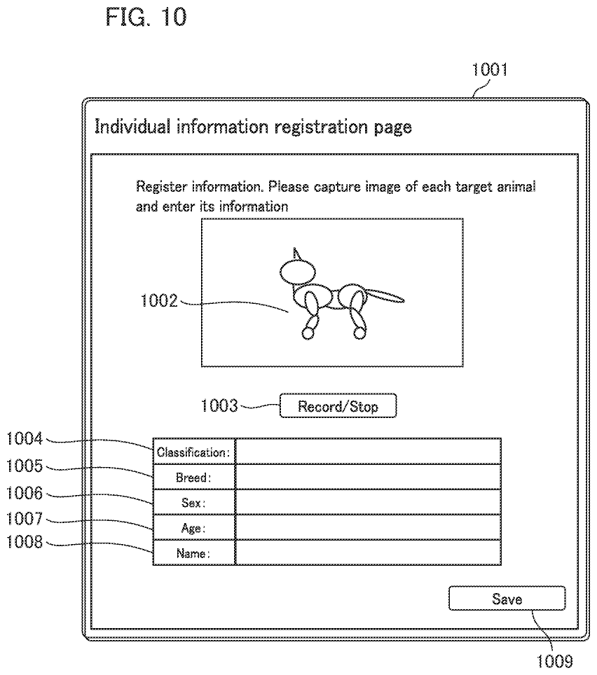

FIG. 10 is a diagram showing an example of the individual information registration page for registering individual information for individual identification in the second embodiment. The individual information registration page 1001 is displayed on the display unit 228 of the local terminal 103 by the UI display unit 308 of the local terminal 103. The individual information registration page 1001 includes a moving image display region 1002 and an individual information input form (a classification input form 1004 to a name input form 1008).

The moving image display region 1002 is a region configured to display moving image data of the individual to be registered. The moving image data of the target individual may be selected from the moving image data stored in the local terminal 103, or moving image data may be obtained by newly capturing a target individual by the video camera 102. A recording button 1003 is a button for the capture of a video image being received from a video camera. Recording is started by a press of the recording button 1003, and is stopped and saved as video data when the button is pressed again during recording. Note that in the present embodiment, although an example in which moving image data is used to register individual information for individual identification is explained, image data may also be used.

The classification input form 1004 is a form into which the classification type of animal that is the target individual of the disease name evaluation is input. A breed input form 1005 is a form to further input what breed of animal species the individual is from among the animal species that was input in the classification input form 1004. A sex input form 1006 is a form to input the sex (female or male) of the individual. The age input form 1007 is a form to input the age of the individual. The name input form 1007 is a form to input the name of the individual. When the save button 1009 is pressed by the user, an individual information registration request for identification that includes individual information and moving image data that has been input in each input form (classification input form 1004 to name input form 1008) is transmitted from the local terminal 103 to the evaluation server 104. Note that the fields of the input form are not limited thereto. For example, there may be a field for the input of information such as medical history, whether or not the animal is pregnant, and the like.

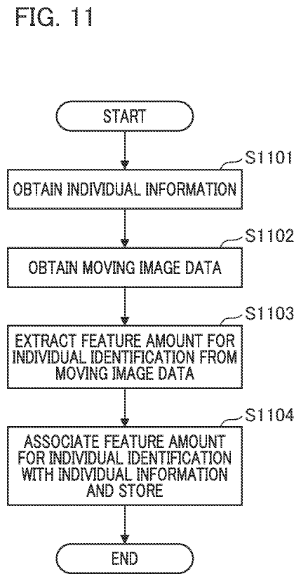

Next, the individual information registration processing in the evaluation server 104 that has received the individual information registration request from the local terminal 103 will be explained. FIG. 11 is a flowchart that shows an individual information registration processing for individual identification according to the second embodiment. Note that the individual information registration process is performed for one animal (one head) at a time. Each of the processes shown in FIG. 11 is realized by the CPU 202 and GPU 209 of the evaluation server 104 loading a program that is stored on the ROM 403 or the HDD 205 on the RAM 204, and executing the program.

The individual information registration processing in the evaluation server 104 is started by receiving an individual information registration request from the local terminal 103. The individual information registration request includes individual information and moving image data. In step S1101, the learning unit 315 of the evaluation server 104 that has received the individual information registration request from the local terminal 103 obtains individual information from the individual information registration request. Individual information is the information that has been input into each input form (classification input form 1004 to name input form 1008) on the individual information registration page 1001. Table 7 is an example of individual information in the second embodiment.

TABLE 7

| | Individ- | Classi- | | | | |
|---|---|---|---|---|---|---|
| User ID | ual ID | fication | Breed | Sex | Age | Name |
| User001 | neko001 | Cat | Tortoiseshell | Male | 5 | Neko A |

In addition to the information in the individual information (Table 1) of the first embodiment, the individual information of the second embodiment further includes a user ID for the unique identification of a user and a name of the individual.

In step S1102, the learning unit 315 obtains moving image data from the individual information registration request. Note that in the present embodiment, although an example in which a feature amount for individual identification is extracted from moving image data will be explained, the present invention is not limited thereto, and in step S1102, image data may be obtained and a feature amount for individual identification extracted from the image data. In step S1103, the learning unit 315 extracts a feature amount for individual identification from the moving image data. For example, the learning unit 315 obtains the classification of an individual from the individual information and extracts a feature amount for individual identification from the moving image data by using a method corresponding to the classification of the animal. Note that a procedure and method for extracting a feature amount for individual identification of an animal may also be used to extract a feature amount of an animal from moving image data or image data by using a different method.

In step S1104, the data storage unit 313 stores the feature amount for individual identification extracted in S1003 on the HDD 205 in association with the individual information obtained in step S1101. Note that in second embodiment, individual information is managed on a per-user basis as shown in Table 8. Table 8 is an example of a list of individual information in the second embodiment.

TABLE 8

| | Individ- | Classi- | | | | |
|---|---|---|---|---|---|---|
| User ID | ual ID | fication | Breed | Sex | Age | Name |
| User001 | neko001 | Cat | Tortoiseshell | Male | 5 | NekoA |
| | neko002 | Cat | Crossbreed | Female | 3 | NekoB |
| | neko003 | Cat | British | Male | 3 | NekoC |
| | | | shorthair | | | |

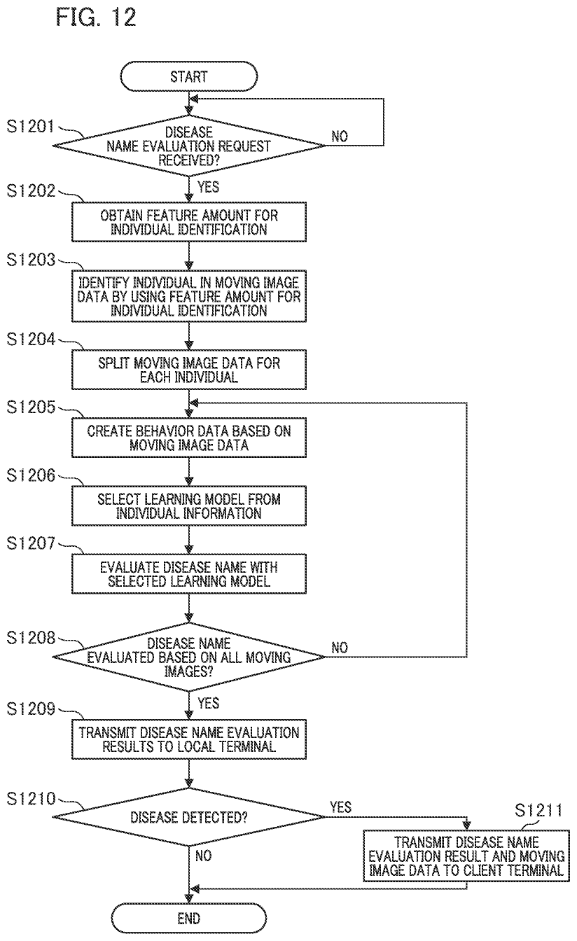

FIG. 12 is a flowchart that shows a disease name evaluation processing in the second embodiment. Each of the processes shown in FIG. 12 is implemented by the CPU 202 and the GPU 209 of the evaluation server 104 loading a program that is stored on the ROM 403 or the HDD 205 onto the RAM 204 and executing the program.

In step S1201, the data reception unit 318 determines whether a disease name evaluation request that includes the user ID of the user managing the individual that is the target of the disease name evaluation and moving image data that is the target of the disease name evaluation has been received from the local terminal 103. Note that in the first embodiment, individual information was included in the disease name evaluation request. However, in the second embodiment, because individual information for individual identification is registered in advance in the evaluation server 104, the disease name evaluation request may not include individual information. In a case in which a disease name evaluation request is received, the processing proceeds to step S1202. In contrast, if no disease name evaluation request has been received, the processing waits until one is received.

In step S1202, the evaluation unit 316 obtains the feature amount for individual identification associated with the user ID included in the disease name evaluation request from HDD 205. In step S1203, the evaluation unit 316 identifies the individual in the moving image data included in the disease name evaluation request by using the feature amount for individual identification obtained in step S1202. In step S1204, the evaluation unit 316 divides the moving image by each identified individual. At this time, an individual that could not be identified due to such reasons as the individual information not being registered is excluded. In a case in which there are no individuals identified, the data processed in steps S1105 to S1107 becomes zero.

In step S1205, the evaluation unit 316 creates behavior data for each individual from the moving image data divided for each individual. In step S1206, the evaluation unit 316 selects a learning model to be used in the evaluation process based on the individual information. In step S1207, the evaluation unit 316 performs disease name evaluation processing by using the learning model selected in step S1206 and the behavior data of the relevant individual, and calculates the disease name evaluation result. Note that because the processing of steps S1205 through S1207 is similar to that of steps S802 through S804 of the first embodiment, a detailed description thereof will be omitted.

In step S1208, the evaluation unit 316 determines whether the disease name evaluation processing has been performed on all of the divided moving image data for each individual. In a case in which the disease name evaluation processing has been performed for all moving image data, the processing proceeds to step S1209. In contrast, in a case in which there is moving image data for which the disease name evaluation processing has not been performed, the processing returns to step S1105 and the disease name evaluation process is performed for the remaining moving image data.

In step S1209, the data transmission unit 317 transmits the disease name evaluation result to the local terminal 103. In step S1210, the data transmission unit 317 uses the disease name evaluation to determine whether or not a disease is detected. In a case in which a disease has not been detected, the disease name evaluation processing is terminated. In contrast, in a case in which a disease is detected, the processing proceeds to step S1211. In step S1211, the data transmission unit 317 transmits the moving image data of the individual and the disease name evaluation results to the client terminal 105. The client terminal 105 that received the moving image data and disease name evaluation results from the evaluation server 104 displays the disease name notification page 901 on the display unit 238, and notifies the user of the disease name evaluation result.

As explained above, according to the present embodiment, even in a case in which a plurality of animals are captured in the moving image data, it will be possible to detect the onset of disease in each individual animal without the need to visit the place where the animal is located.

### Other Embodiments

Embodiments of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)TM), a flash memory device, a memory card, and the like.

A processor or circuitry may include a graphics processing unit (GPU), or a field programmable gateway (FPGA). In addition, the processor or circuitry may also include a digital signal processor (DSP), data flow processor (DFP), or neural processing unit (NPU).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2021-140655, filed Aug. 31 2021, which is hereby incorporated by reference wherein in its entirety.

What is claimed is:

1. A system that includes an evaluation server that uses a learning model to perform an evaluation of a disease name of an animal, a first information processing device configured to request an evaluation of the name of a disease from the evaluation server, and a second information processing device configured to display a result that has been evaluated by the evaluation server, the system comprising:
the first information processing device comprising:
a memory storing instructions; and
a processor executing the instructions causing the first information processing device to:
transmit a request for a disease name evaluation that includes moving image data in which an animal is captured to the evaluation server, the evaluation server comprising:
a memory storing instructions; and
a processor executing the instructions causing the evaluation server to:
evaluate, in response to receiving a disease name evaluation request from the first information processing device, a disease name from the moving image data by use of a learning model selected based on individual information of an individual that is a target of disease name evaluation, and
notify the second information processing device of a result of an evaluation by the evaluation unit,
the second information processing device comprising:
a memory storing instructions; and
a processor executing the instructions causing the second information processing device to:
display a notification page configured to include the result of the evaluation obtained from the evaluation server.

2. The system according to claim 1,
wherein the processor of the evaluation server further executes an instruction causing the evaluation server to generate a learning model for disease name evaluation based on moving image data by machine learning that uses moving image data for learning and a disease name.

3. The system according to claim 2,
wherein the processor of the evaluation server displays the result of the evaluation on the notification page and moving image data, and a UI for the input of feedback information with respect to the result of the evaluation, and
wherein the processor of second information processing device further executes an instruction to cause the evaluation server to transmit feedback information with respect to the result of the evaluation that has been input by a user on the notification page to the evaluation server.

4. The system according to claim 3,
wherein the processor of the evaluation server generates or updates a learning model by machine learning that uses moving image data in which disease name evaluation is performed, and the feedback information that has been obtained from the second information processing device that corresponds to the moving image data.

5. The system according to claim 3,
wherein the feedback information is a disease name of a result of a diagnosis performed by an specialist that is input by a user in the notification page.

6. The system according to claim 1,
wherein the processor of the evaluation server extracts behavior data that is time-series data of position information of each portion of a diagnosis target from the moving image data, and evaluates a disease name based on the behavior data by use of a selected learning model.

7. The system according to claim 1,
wherein the individual information includes at least one of a classification, a breed, a sex, and an age of an individual.

8. The system according to claim 1,
wherein the request includes moving image data in which an animal is captured and individual information of the individual captured animal, and
wherein the processor of the evaluation server selects a learning model based on the individual information that is included in the evaluation request.

9. The system according to claim 1,
wherein the processor of the first information processing device further executes an instruction causing the first information processing device to transmit a registration request of information for individual identification that includes moving image data that is captured of one animal at a time and individual information of each individual captured to the evaluation server,
wherein the processor of evaluation server further executes an instruction causing the evaluation server to, in response to receipt of the registration request, extract a feature amount for the identification of an individual from moving image data that is included in the registration request, and register the feature amount and the individual information in association with each other, and
wherein the processor of the evaluation server divides the moving image data for each individual by identifying an individual in moving image data by using the feature amount, and evaluates a disease name based on the divided moving image data.

10. A control method for a system which includes an evaluation server that uses a learning model to perform an evaluation of a disease name of an animal, a first information processing device configured to request an evaluation of the name of a disease from the evaluation server, and a second information processing device configured to display a result evaluated by the evaluation server, the method comprising:
transmitting request for a disease name evaluation that includes moving image data in which an animal is captured to the evaluation server from the first information processing device,
evaluating on the evaluation server, in response to receiving a disease name evaluation request from the first information processing device, a disease name based on the moving image data by using a learning model selected based on individual information of an individual that is a target of disease name evaluation,
notifying the result of the evaluation that was evaluated from the evaluation server to the second information processing device, and
displaying, in the second information processing device, a notification page configured to include the result of the evaluation obtained from the evaluation server.
